(19) United States
(12) Patent Application Publication
(10) Pub. No.: US 2016/0038277 A1
(43) Pub. Date: Feb. 11, 2016

(54) Optical Surface Systems and Methods for Treatment of Presbyopia and Other Vision Conditions

(71) Applicant: AMO Development, LLC, Santa Ana, CA (US)

(72) Inventors: Guang-ming Dai, Fremont, CA (US); Henk Weeber, Groningen (NL); Huawei Zhao, Irvine, CA (US)

(21) Appl. No.: 14/819,123

(22) Filed: Aug. 5, 2015

Related U.S. Application Data
(60) Provisional application No. 62/035,874, filed on Aug. 11, 2014.

(51) Int. Cl. A61F 2/16; G06F 17/10; G06F 17/50

(52) U.S. Cl. CPC A61F 2/16; A61F 2/1662; G06F 17/50; G06F 17/10

(57) ABSTRACT

Systems and methods for generating an treatment shape for treating an eye of a patient involve obtaining a treatment effect corresponding to an operator influence profile, obtaining a base shape, producing a set of adjusted shapes that are based on a random fluctuation of the base shape and the treatment effect, evaluating the adjusted shapes of the set according to a merit function, selecting one of the adjusted shapes based on the evaluation, and generating the treatment shape based on the selected shape.

### CROSS-REFERENCES TO RELATED APPLICATIONS

This application is a non-provisional of and claims the benefit of priority to U.S. Provisional Patent Application No. 62/035,874 filed Aug. 11, 2014, the entire contents of which is incorporated herein by reference.

### BACKGROUND OF THE INVENTION

Embodiments of the present invention relate to vision treatment systems and methods, and in particular to treatment shape generation techniques for use with intraocular lens devices and methods, and other vision treatment modalities.

Intraocular lenses are commonly used in the field of ophthalmology for treating cataracts and other vision conditions. Often, the patient's natural crystalline lens is removed, and replaced with a synthetic or manufactured intraocular lens (IOL). In some cases, the IOL is implanted while the patient's natural lens is left in place. Typically, the IOL is placed in the capsular bag of the patient's eye. Currently available IOLs are useful for treating any of a variety of conditions, including for example aphakia, astigmatism, myopia, and the like. In many cases, a surgeon or operator uses an implantation system to administer the IOL to the patient, for example by folding and assisting with the insertion of the IOL into the capsular bag via an incision in the bag.

Although these and other proposed treatment devices and methods may provide real benefits to patients in need thereof, still further advances would be desirable. For example, there continues to be a need for improved treatment systems and methods that provide enhanced accuracy of treatment. Embodiments of the present invention provide solutions that address certain inefficiencies or shortcomings which may be associated with known techniques, and hence provide answers to at least some of these outstanding needs.

### BRIEF SUMMARY OF THE INVENTION

During an intraocular lens implantation procedure, a surgeon may place marks on the horizontal and/or vertical axis of the lens for reference purposes, and use them as a guide in order to achieve a desired implantation orientation for the lens (e.g. to ensure the lens is positioned at the desired rotational angle in the patient eye). The surgeon may also take steps to ensure that the lens plane is perpendicular to the optical axis or visual axis of the eye, or is otherwise oriented as desired relative to the optical axis or visual axis of the eye. In some instances, such positioning can be performed to ensure that light passes through the lens at the desired angle (e.g. perpendicular to the lens plane) so as to minimize or control for deformation of the image or light rays.

Despite such care, various complications may arise related to misplacement of the lens during the implantation procedure. Relatedly, currently used optical designs are often very sensitive to lens misplacement such as rotational error, collective tilt error, and the like. Embodiments of the present invention encompass systems and methods for developing optical treatment shapes that tolerate such intraoperative misplacement effects by providing excellent vision results in spite of such misplacement. For example, such treatment shapes can be implanted in offset orientations or positions relate to a target orientation, and yet the misalignment or misplacement does little to diminish the improvement in vision conferred by the lens. In some cases, optical treatment shapes can be developed to tolerate postoperative changes in rotational error, collective tilt, error, and the like.

Embodiments of the present invention encompass techniques for also rotation, tip, and tilt in relation to contact lenses, spectacle lenses, refractive surgery, and other vision treatment modalities such as inlays, conductive keratoplasty, and the like. Such lenses or treatments can be developed to treat any of a variety of vision conditions, such as myopia, presbyopia, hyperopia, astigmatism, and the like.

In a first aspect, embodiments of the present invention encompass methods for generating an intraocular treatment lens shape for treating an eye of a patient. Exemplary methods can include obtaining an intraocular lens implantation effect corresponding to an operator influence profile, obtaining an intraocular lens base shape, and producing a set of adjusted intraocular lens shapes, where individual adjusted shapes of the set are based on a random fluctuation of the base shape and the set is based on the implantation effect. Methods can also include evaluating the adjusted shapes of the set according to a merit function, selecting one of the adjusted shapes based on the evaluation, and generating the intraocular lens treatment shape based on the selected shape. In some cases, the intraocular lens implantation effect includes a rotation effect, a tilt effect, or both. In some cases, the intraocular lens implantation effect includes a rotation effect. In some cases, the rotation effect corresponds to a rotational range. In some cases, the rotational range corresponds to a 20 degree arc of angular rotation. In some cases, the rotational range includes a negative limit corresponding to a maximum negative intraoperative or postoperative rotation of the lens. In some cases, the rotational range includes a positive limit corresponding to a maximum positive intraoperative or postoperative rotation of the lens. In some cases, the negative limit is −10 degrees relative to an intended lens orientation. In some cases, the positive limit is +10 degrees relative to the intended lens orientation. In some cases, the intraocular lens implantation effect includes a tilt effect. In some cases, the tilt effect corresponds to a tilt range. In some cases, the tilt range corresponds to a 15 degree arc of angular tilt. In some cases, the tilt range includes a negative limit corresponding to a maximum negative intraoperative or postoperative tilt of the lens. In some cases, the tilt range includes a positive limit corresponding to a maximum positive intraoperative or postoperative tilt of the lens. In some cases, the negative limit is −7.5 degrees relative to an intended lens orientation. In some cases, the positive limit is +7.5 degrees relative to the intended lens orientation. In some cases, the operator influence profile corresponds to a physician population. In some cases, the merit function includes a Strehl Ratio (SR) parameter, a modulation transfer function (MTF) parameter, a point spread function (PSF), an encircled energy (EE) parameter, a volume under MTF surface (MTFV) parameter, a compound modulation transfer function (CMTF) parameter, or a contrast sensitivity (CS) parameter.

In another aspect, embodiments of the present invention encompass systems for generating an intraocular treatment lens shape for treating an eye of a patient. Exemplary systems can include a processor and a tangible non-transitory computer readable medium. The computer readable medium can include a computer application that, when executed by the processor, causes the processor to access an intraocular lens implantation effect corresponding to an operator influence profile, to access an intraocular lens base shape, and to produce a set of adjusted intraocular lens shapes, where individual adjusted shapes of the set are produced based on a random fluctuation of the base shape and the set is produced based on the implantation effect. The computer application, when executed by the processor, can also the processor to evaluate the adjusted shapes of the set according to a merit function, to select one of the adjusted shapes based on the evaluation, and to generate the intraocular lens treatment shape based on the selected shape. In some cases, the intraocular lens implantation effect includes a rotation effect, a tilt effect, or both. In some cases, the intraocular lens implantation effect includes a rotation effect. In some cases, the rotation effect corresponds to a rotational range. In some cases, the rotational range corresponds to a 20 degree arc of angular rotation. In some cases, the rotational range includes a negative limit corresponding to a maximum negative intraoperative or postoperative rotation of the lens. In some cases, the rotational range includes a positive limit corresponding to a maximum positive intraoperative or postoperative rotation of the lens. In some cases, the negative limit is −10 degrees relative to an intended lens orientation. In some cases, the positive limit is +10 degrees relative to the intended lens orientation. In some cases, the intraocular lens implantation effect includes a tilt effect. In some cases, the tilt effect corresponds to a tilt range. In some cases, the tilt range corresponds to a 15 degree arc of angular tilt. In some cases, the tilt range includes a negative limit corresponding to a maximum negative intraoperative or postoperative tilt of the lens. In some cases, the tilt range includes a positive limit corresponding to a maximum positive intraoperative or postoperative tilt of the lens. In some cases, the negative limit is −7.5 degrees relative to an intended lens orientation. In some cases, the positive limit is +7.5 degrees relative to the intended lens orientation. In some cases, the operator influence profile corresponds to a physician population. In some cases, the merit function includes a Strehl Ratio (SR) parameter, a modulation transfer function (MTF) parameter, a point spread function (PSF), an encircled energy (EE) parameter, a volume under MTF surface (MTFV) parameter, a compound modulation transfer function (CMTF) parameter, or a contrast sensitivity (CS) parameter.

In still another aspect, embodiments of the present invention encompass computer products for generating intraocular lens treatment shapes. Exemplary computer products be embodied on a tangible non-transitory computer readable storage medium, and can include code for accessing an intraocular lens implantation effect corresponding to an operator influence profile, code for accessing an intraocular lens base shape, and code for producing a set of adjusted intraocular lens shapes, where individual adjusted shapes of the set are produced based on a random fluctuation of the base shape and the set is produced based on the implantation effect. Computer products can also include code for evaluating the adjusted shapes of the set according to a merit function, code for selecting one of the adjusted shapes based on the evaluation, and code for generating the intraocular lens treatment shape based on the selected shape. In some cases, the intraocular lens implantation effect includes a rotation effect, a tilt effect, or both. In some cases, the intraocular lens implantation effect includes a rotation effect. In some cases, the rotation effect corresponds to a rotational range. In some cases, the rotational range corresponds to a 20 degree arc of angular rotation. In some cases, the rotational range includes a negative limit corresponding to a maximum negative intraoperative or postoperative rotation of the lens. In some cases, the rotational range includes a positive limit corresponding to a maximum positive intraoperative or postoperative rotation of the lens. In some cases, the negative limit is −10 degrees relative to an intended lens orientation. In some cases, the positive limit is +10 degrees relative to the intended lens orientation. In some cases, the intraocular lens implantation effect includes a tilt effect. In some cases, the tilt effect corresponds to a tilt range. In some cases, the tilt range corresponds to a 15 degree arc of angular tilt. In some cases, the tilt range includes a negative limit corresponding to a maximum negative intraoperative or postoperative tilt of the lens. In some cases, the tilt range includes a positive limit corresponding to a maximum positive intraoperative or postoperative tilt of the lens. In some cases, the negative limit is −7.5 degrees relative to an intended lens orientation. In some cases, the positive limit is +7.5 degrees relative to the intended lens orientation. In some cases, the operator influence profile corresponds to a physician population. In some cases, the merit function includes a Strehl Ratio (SR) parameter, a modulation transfer function (MTF) parameter, a point spread function (PSF), an encircled energy (EE) parameter, a volume under MTF surface (MTFV) parameter, a compound modulation transfer function (CMTF) parameter, or a contrast sensitivity (CS) parameter.

For a fuller understanding of the nature and advantages of the present invention, reference should be had to the ensuing detailed description taken in conjunction with the accompanying drawings.

### BRIEF DESCRIPTION OF THE DRAWINGS

FIG. 1 illustrates a laser ablation system according to embodiments of the present invention.

FIG. 2 illustrates a simplified computer system according to embodiments of the present invention.

FIG. 3 illustrates a wavefront measurement system according to embodiments of the present invention.

FIG. 3A illustrates another wavefront measurement system according to embodiments of the present invention.

FIG. 4 depicts rotation aspects of an implanted intraocular lens, provided by a front view of the eye.

FIG. 5 depicts tilt aspects of an implanted intraocular lens, provided by an overhead view of the eye.

FIG. 6 depicts tip aspects of an implanted intraocular lens, provided by a side view of the eye.

FIG. 7 depicts aspects of an intraocular lens and related potential intraoperative effects associated with implantation of the lens (or postoperative effects), according to embodiments of the present invention.

FIG. 8 depicts aspects of a vision treatment generation process according to embodiments of the present invention.

FIG. 9 depicts aspects of a vision treatment generation process according to embodiments of the present invention.

### DETAILED DESCRIPTION OF THE INVENTION

Embodiments of the present invention encompass systems and methods for generating vision treatment shapes that tolerate, for example, various degrees of intraoperative rotation, tip, and tilt associated with implantation of an intraocular lens. Relatedly, such shapes can tolerate various degrees of rotation, tip, and tilt associate with a variety of optical treatment modalities. In some cases, the vision treatment shapes can tolerate various degrees of postoperative rotation, tip, and tilt that may occur following implantation of an intraocular lens. Hence, such vision treatment shapes or designs can be administered to a patient, with minimal impact to the visual effect or optical effect imparted by the treatment shape. For example, embodiments of the present invention encompass techniques for developing a lens design that provides good vision for the patient or good optical performance, for example as evaluated by a merit function or an optical metric such as CMTF, even though the lens may be misplaced (e.g. off-axis, rotated, tipped, or tilted) relative to a target orientation.

Embodiments of the present invention can be readily adapted for use with existing laser systems and other optical treatment devices. Although certain system, software, and method embodiments of the present invention are described primarily in the context of a laser eye surgery system, it should be understood that embodiments of the present invention may be adapted for use in alternative eye treatment procedures, systems, or modalities, such as spectacle lenses, intraocular lenses, accommodating IOLs, contact lenses, corneal ring implants, collagenous corneal tissue thermal remodeling, corneal inlays, corneal onlays, other corneal implants or grafts, and the like. Relatedly, systems, software, and methods according to embodiments of the present invention are well suited for customizing any of these treatment modalities to a specific patient. Thus, for example, embodiments encompass custom intraocular lenses, custom contact lenses, custom corneal implants, and the like, which can be configured to treat or ameliorate any of a variety of vision conditions in a particular patient based on their unique ocular characteristics or anatomy. Additionally, the ablation target or target shape may be implemented via other non-ablative laser therapies, such as laser-incised custom lenticule shapes and subsequent extraction and laser-based corneal incision patterns.

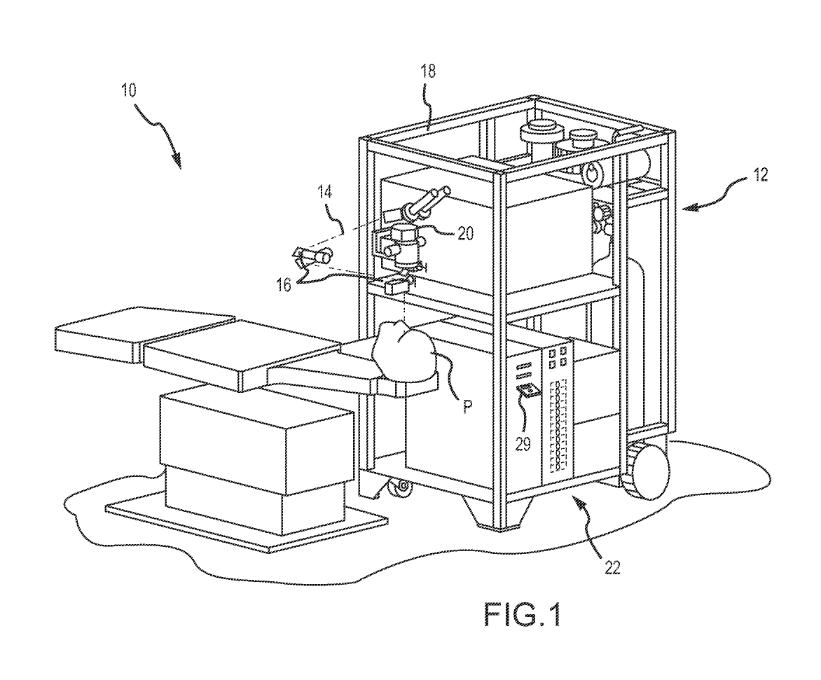

Turning now to the drawings, FIG. 1 illustrates a laser eye surgery system 10 of the present invention, including a laser 12 that produces a laser beam 14. Laser 12 is optically coupled to laser delivery optics 16, which directs laser beam 14 to an eye E of patient P. A delivery optics support structure (not shown here for clarity) extends from a frame 18 supporting laser 12. A microscope 20 is mounted on the delivery optics support structure, the microscope often being used to image a cornea of eye E.

Laser 12 generally comprises an excimer laser, ideally comprising an argon-fluorine laser producing pulses of laser light having a wavelength of approximately 193 nm. Laser 12 will preferably be designed to provide a feedback stabilized fluence at the patient's eye, delivered via delivery optics 16. The present invention may also be useful with alternative sources of ultraviolet or infrared radiation, particularly those adapted to controllably ablate the corneal tissue without causing significant damage to adjacent and/or underlying tissues of the eye. Such sources include, but are not limited to, solid state lasers and other devices which can generate energy in the ultraviolet wavelength between about 185 and 205 nm and/or those which utilize frequency-multiplying techniques. Hence, although an excimer laser is the illustrative source of an ablating beam, other lasers may be used in the present invention.

Laser system 10 will generally include a computer or programmable processor 22. Processor 22 may comprise (or interface with) a conventional PC system including the standard user interface devices such as a keyboard, a display monitor, and the like. Processor 22 will typically include an input device such as a magnetic or optical disk drive, an internet connection, or the like. Such input devices will often be used to download a computer executable code from a tangible storage media 29 embodying any of the methods of the present invention. Tangible storage media 29 may take the form of a floppy disk, an optical disk, a data tape, a volatile or non-volatile memory, RAM, or the like, and the processor 22 will include the memory boards and other standard components of modern computer systems for storing and executing this code. Tangible storage media 29 may optionally embody wavefront sensor data, wavefront gradients, a wavefront elevation map, a treatment map, a corneal elevation map, and/or an ablation table. While tangible storage media 29 will often be used directly in cooperation with an input device of processor 22, the storage media may also be remotely operatively coupled with processor by means of network connections such as the internet, and by wireless methods such as infrared, Bluetooth, or the like.

Laser 12 and delivery optics 16 will generally direct laser beam 14 to the eye of patient P under the direction of a computer 22. Computer 22 will often selectively adjust laser beam 14 to expose portions of the cornea to the pulses of laser energy so as to effect a predetermined sculpting of the cornea and alter the refractive characteristics of the eye. In many embodiments, both laser beam 14 and the laser delivery optical system 16 will be under computer control of processor 22 to effect the desired laser sculpting process, with the processor effecting (and optionally modifying) the pattern of laser pulses. The pattern of pulses may by summarized in machine readable data of tangible storage media 29 in the form of a treatment table, and the treatment table may be adjusted according to feedback input into processor 22 from an automated image analysis system in response to feedback data provided from an ablation monitoring system feedback system. Optionally, the feedback may be manually entered into the processor by a system operator. Such feedback might be provided by integrating the wavefront measurement system described below with the laser treatment system 10, and processor 22 may continue and/or terminate a sculpting treatment in response to the feedback, and may optionally also modify the planned sculpting based at least in part on the feedback. Measurement systems are further described in U.S. Pat. No. 6,315,413, the full disclosure of which is incorporated herein by reference.

Laser beam 14 may be adjusted to produce the desired sculpting using a variety of alternative mechanisms. The laser beam 14 may be selectively limited using one or more variable apertures. An exemplary variable aperture system having a variable iris and a variable width slit is described in U.S. Pat. No. 5,713,892, the full disclosure of which is incorporated herein by reference. The laser beam may also be tailored by varying the size and offset of the laser spot from an axis of the eye, as described in U.S. Pat. Nos. 5,683,379, 6,203,539, and 6,331,177, the full disclosures of which are incorporated herein by reference.

Still further alternatives are possible, including scanning of the laser beam over the surface of the eye and controlling the number of pulses and/or dwell time at each location, as described, for example, by U.S. Pat. No. 4,665,913, the full disclosure of which is incorporated herein by reference; using masks in the optical path of laser beam 14 which ablate to vary the profile of the beam incident on the cornea, as described in U.S. Pat. No. 5,807,379, the full disclosure of which is incorporated herein by reference; hybrid profile-scanning systems in which a variable size beam (typically controlled by a variable width slit and/or variable diameter iris diaphragm) is scanned across the cornea; or the like. The computer programs and control methodology for these laser pattern tailoring techniques are well described in the patent literature.

Additional components and subsystems may be included with laser system 10, as should be understood by those of skill in the art. For example, spatial and/or temporal integrators may be included to control the distribution of energy within the laser beam, as described in U.S. Pat. No. 5,646,791, the full disclosure of which is incorporated herein by reference. Ablation effluent evacuators/filters, aspirators, and other ancillary components of the laser surgery system are known in the art. Further details of suitable systems for performing a laser ablation procedure can be found in commonly assigned U.S. Pat. Nos. 4,665,913, 4,669,466, 4,732,148, 4,770,172, 4,773,414, 5,207,668, 5,108,388, 5,219,343, 5,646,791 and 5,163,934, the complete disclosures of which are incorporated herein by reference. Suitable systems also include commercially available refractive laser systems such as those manufactured and/or sold by Alcon, Bausch & Lomb, Nidek, WaveLight, LaserSight, Schwind, Zeiss-Meditec, and the like. Basis data can be further characterized for particular lasers or operating conditions, by taking into account localized environmental variables such as temperature, humidity, airflow, and aspiration.

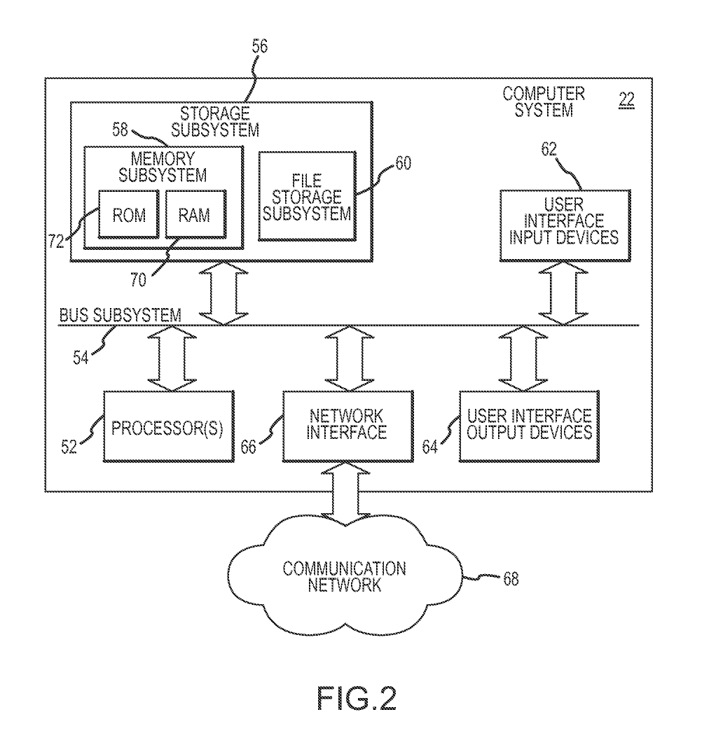

FIG. 2 is a simplified block diagram of an exemplary computer system 22 that may be used by the laser surgical system 10 of the present invention. Computer system 22 typically includes at least one processor 52 which may communicate with a number of peripheral devices via a bus subsystem 54. These peripheral devices may include a storage subsystem 56, comprising a memory subsystem 58 and a file storage subsystem 60, user interface input devices 62, user interface output devices 64, and a network interface subsystem 66. Network interface subsystem 66 provides an interface to outside networks 68 and/or other devices, such as the wavefront measurement system 30.

User interface input devices 62 may include a keyboard, pointing devices such as a mouse, trackball, touch pad, or graphics tablet, a scanner, foot pedals, a joystick, a touchscreen incorporated into the display, audio input devices such as voice recognition systems, microphones, and other types of input devices. User input devices 62 will often be used to download a computer executable code from a tangible storage media 29 embodying any of the methods of the present invention. In general, use of the term “input device” is intended to include a variety of conventional and proprietary devices and ways to input information into computer system 22.

User interface output devices 64 may include a display subsystem, a printer, a fax machine, or non-visual displays such as audio output devices. The display subsystem may be a cathode ray tube (CRT), a flat-panel device such as a liquid crystal display (LCD), a projection device, or the like. The display subsystem may also provide a non-visual display such as via audio output devices. In general, use of the term “output device” is intended to include a variety of conventional and proprietary devices and ways to output information from computer system 22 to a user.

Storage subsystem 56 can store the basic programming and data constructs that provide the functionality of the various embodiments of the present invention. For example, a database and modules implementing the functionality of the methods of the present invention, as described herein, may be stored in storage subsystem 56. These software modules are generally executed by processor 52. In a distributed environment, the software modules may be stored on a plurality of computer systems and executed by processors of the plurality of computer systems. Storage subsystem 56 typically comprises memory subsystem 58 and file storage subsystem 60.

Memory subsystem 58 typically includes a number of memories including a main random access memory (RAM) 70 for storage of instructions and data during program execution and a read only memory (ROM) 72 in which fixed instructions are stored. File storage subsystem 60 provides persistent (non-volatile) storage for program and data files, and may include tangible storage media 29 (FIG. 1) which may optionally embody wavefront sensor data, wavefront gradients, a wavefront elevation map, a treatment map, and/or an ablation table. File storage subsystem 60 may include a hard disk drive, a floppy disk drive along with associated removable media, a Compact Digital Read Only Memory (CD-ROM) drive, an optical drive, DVD, CD-R, CD-RW, solid-state removable memory, and/or other removable media cartridges or disks. One or more of the drives may be located at remote locations on other connected computers at other sites coupled to computer system 22. The modules implementing the functionality of the present invention may be stored by file storage subsystem 60.

Bus subsystem 54 provides a mechanism for letting the various components and subsystems of computer system 22 communicate with each other as intended. The various subsystems and components of computer system 22 need not be at the same physical location but may be distributed at various locations within a distributed network. Although bus subsystem 54 is shown schematically as a single bus, alternate embodiments of the bus subsystem may utilize multiple busses.

Computer system 22 itself can be of varying types including a personal computer, a portable computer, a workstation, a computer terminal, a network computer, a control system in a wavefront measurement system or laser surgical system, a mainframe, or any other data processing system. Due to the ever-changing nature of computers and networks, the description of computer system 22 depicted in FIG. 2 is intended only as a specific example for purposes of illustrating one embodiment of the present invention. Many other configurations of computer system 22 are possible having more or less components than the computer system depicted in FIG. 2.

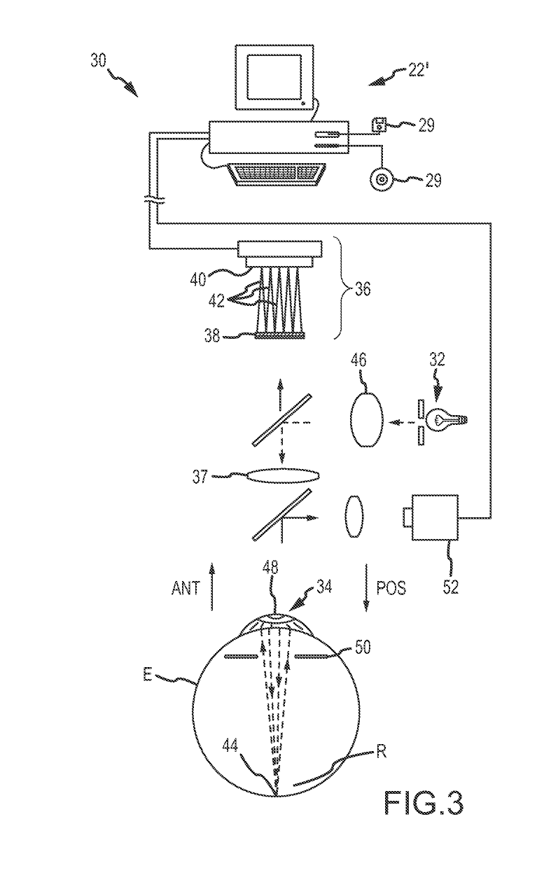

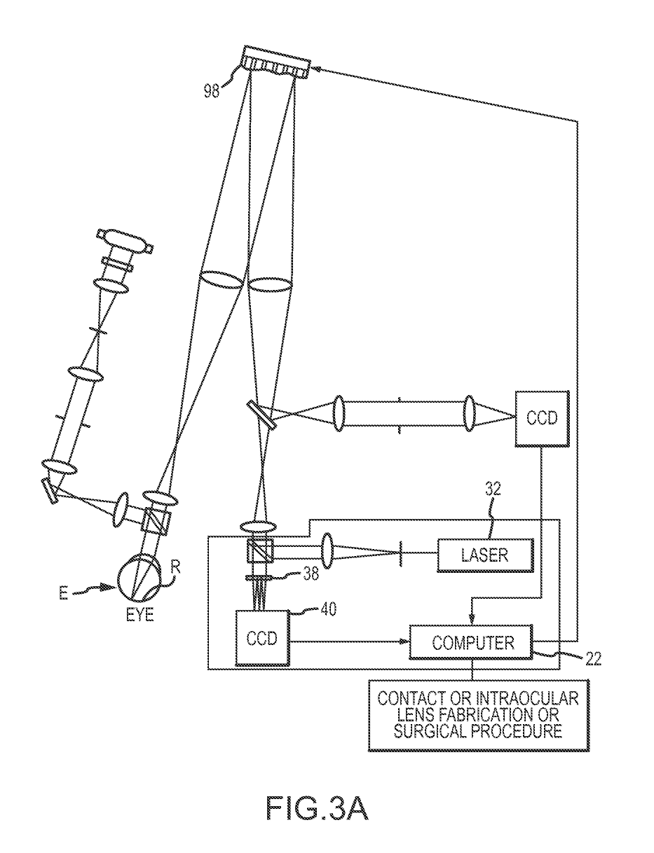

Referring now to FIG. 3, one embodiment of a wavefront measurement system 30 is schematically illustrated in simplified form. In very general terms, wavefront measurement system 30 is configured to sense local slopes of a gradient map exiting the patient's eye. Devices based on the Hartmann-Shack principle generally include a lenslet array to sample the gradient map uniformly over an aperture, which is typically the exit pupil of the eye. Thereafter, the local slopes of the gradient map are analyzed so as to reconstruct the wavefront surface or map.

More specifically, one wavefront measurement system 30 includes an image source 32, such as a laser, which projects a source image through optical tissues 34 of eye E so as to form an image 44 upon a surface of retina R. The image from retina R is transmitted by the optical system of the eye (e.g., optical tissues 34) and imaged onto a wavefront sensor 36 by system optics 37. The wavefront sensor 36 communicates signals to a computer system 22′ for measurement of the optical errors in the optical tissues 34 and/or determination of an optical tissue ablation treatment program. Computer 22′ may include the same or similar hardware as the computer system 22 illustrated in FIGS. 1 and 2. Computer system 22′ may be in communication with computer system 22 that directs the laser surgery system 10, or some or all of the components of computer system 22, 22′ of the wavefront measurement system 30 and laser surgery system 10 may be combined or separate. If desired, data from wavefront sensor 36 may be transmitted to a laser computer system 22 via tangible media 29, via an I/O port, via an networking connection 66 such as an intranet or the Internet, or the like.

Wavefront sensor 36 generally comprises a lenslet array 38 and an image sensor 40. As the image from retina R is transmitted through optical tissues 34 and imaged onto a surface of image sensor 40 and an image of the eye pupil P is similarly imaged onto a surface of lenslet array 38, the lenslet array separates the transmitted image into an array of beamlets 42, and (in combination with other optical components of the system) images the separated beamlets on the surface of sensor 40. Sensor 40 typically comprises a charged couple device or “CCD,” and senses the characteristics of these individual beamlets, which can be used to determine the characteristics of an associated region of optical tissues 34. In particular, where image 44 comprises a point or small spot of light, a location of the transmitted spot as imaged by a beamlet can directly indicate a local gradient of the associated region of optical tissue.

Eye E generally defines an anterior orientation ANT and a posterior orientation POS. Image source 32 generally projects an image in a posterior orientation through optical tissues 34 onto retina R as indicated in FIG. 3. Optical tissues 34 again transmit image 44 from the retina anteriorly toward wavefront sensor 36. Image 44 actually formed on retina R may be distorted by any imperfections in the eye's optical system when the image source is originally transmitted by optical tissues 34. Optionally, image source projection optics 46 may be configured or adapted to decrease any distortion of image 44.

In some embodiments, image source optics 46 may decrease lower order optical errors by compensating for spherical and/or cylindrical errors of optical tissues 34. Higher order optical errors of the optical tissues may also be compensated through the use of an adaptive optic element, such as a deformable mirror (described below). Use of an image source 32 selected to define a point or small spot at image 44 upon retina R may facilitate the analysis of the data provided by wavefront sensor 36. Distortion of image 44 may be limited by transmitting a source image through a central region 48 of optical tissues 34 which is smaller than a pupil 50, as the central portion of the pupil may be less prone to optical errors than the peripheral portion. Regardless of the particular image source structure, it will be generally be beneficial to have a well-defined and accurately formed image 44 on retina R.

In one embodiment, the wavefront data may be stored in a computer readable medium 29 or a memory of the wavefront sensor system 30 in two separate arrays containing the x and y wavefront gradient values obtained from image spot analysis of the Hartmann-Shack sensor images, plus the x and y pupil center offsets from the nominal center of the Hartmann-Shack lenslet array, as measured by the pupil camera 51 (FIG. 3) image. Such information contains all the available information on the wavefront error of the eye and is sufficient to reconstruct the wavefront or any portion of it. In such embodiments, there is no need to reprocess the Hartmann-Shack image more than once, and the data space required to store the gradient array is not large. For example, to accommodate an image of a pupil with an 8 mm diameter, an array of a 20×20 size (i.e., 400 elements) is often sufficient. As can be appreciated, in other embodiments, the wavefront data may be stored in a memory of the wavefront sensor system in a single array or multiple arrays.

While the methods of the present invention will generally be described with reference to sensing of an image 44, a series of wavefront sensor data readings may be taken. For example, a time series of wavefront data readings may help to provide a more accurate overall determination of the ocular tissue aberrations. As the ocular tissues can vary in shape over a brief period of time, a plurality of temporally separated wavefront sensor measurements can avoid relying on a single snapshot of the optical characteristics as the basis for a refractive correcting procedure. Still further alternatives are also available, including taking wavefront sensor data of the eye with the eye in differing configurations, positions, and/or orientations. For example, a patient will often help maintain alignment of the eye with wavefront measurement system 30 by focusing on a fixation target, as described in U.S. Pat. No. 6,004,313, the full disclosure of which is incorporated herein by reference. By varying a position of the fixation target as described in that reference, optical characteristics of the eye may be determined while the eye accommodates or adapts to image a field of view at a varying distance and/or angles.

The location of the optical axis of the eye may be verified by reference to the data provided from a pupil camera 52. In the exemplary embodiment, a pupil camera 52 images pupil 50 so as to determine a position of the pupil for registration of the wavefront sensor data relative to the optical tissues.

An alternative embodiment of a wavefront measurement system is illustrated in FIG. 3A. The major components of the system of FIG. 3A are similar to those of FIG. 3. Additionally, FIG. 3A includes an adaptive optical element 53 in the form of a deformable mirror. The source image is reflected from deformable mirror 98 during transmission to retina R, and the deformable mirror is also along the optical path used to form the transmitted image between retina R and imaging sensor 40. Deformable mirror 98 can be controllably deformed by computer system 22 to limit distortion of the image formed on the retina or of subsequent images formed of the images formed on the retina, and may enhance the accuracy of the resultant wavefront data. The structure and use of the system of FIG. 3A are more fully described in U.S. Pat. No. 6,095,651, the full disclosure of which is incorporated herein by reference.

The components of an embodiment of a wavefront measurement system for measuring the eye and ablations may comprise elements of a WaveScan® system, available from AMO MANUFACTURING USA, LLC, MILPITAS, Calif. One embodiment includes a WaveScan system with a deformable mirror as described above. An alternate embodiment of a wavefront measuring system is described in U.S. Pat. No. 6,271,915, the full disclosure of which is incorporated herein by reference. It is appreciated that any wavefront aberrometer could be employed for use with the present invention. Relatedly, embodiments of the present invention encompass the implementation of any of a variety of optical instruments provided by AMO WaveFront Sciences, LLC, including the COAS wavefront aberrometer, the ClearWave contact lens aberrometer, the CrystalWave IOL aberrometer, and the like.

Relatedly, embodiments of the present invention encompass the implementation of any of a variety of optical instruments provided by WaveFront Sciences, Inc., including the COAS wavefront aberrometer, the ClearWave contact lens aberrometer, the CrystalWave IOL aberrometer, and the like. Embodiments of the present invention may also involve wavefront measurement schemes such as a Tscherning-based system, which may be provided by Alcon, Inc. Embodiments of the present invention may also involve wavefront measurement schemes such as a ray tracing-based system, which may be provided by Tracey Technologies, Corp.

Ocular wavefront transformation is suitable for use in wavefront optics for vision correction because the pupil size of a human eye often changes due to accommodation or the change of lighting, and because the pupil constriction is commonly not concentric. Certain features of these ocular effects are discussed in, for example, Wilson, M. A. et al., Optom. Vis. Sci., 69:129-136 (1992), Yang, Y. et al., Invest. Ophthal. Vis. Sci., 43:2508-2512 (2002), and Donnenfeld, E. J., Refract. Surg., 20:593-596 (2004). For example, in laser vision correction, the pupil size of an eye is relatively large when an ocular wavefront is captured under an aberrometer. To obtain the entire ocular wavefront, it is often recommended that the ambient light be kept low so as to dilate the pupil size during the wavefront exam. A larger wavefront map can provide surgeons the flexibility for treatment over a smaller zone, because the wavefront information over any smaller zone within a larger zone is known. When a smaller wavefront map is captured, however, it is also useful to devise an accurate treatment over a larger zone. When the patient is under the laser, the pupil size can change due to changes in the ambient light. In many cases, the surgery room is brighter than a wavefront examination room, in particular when the patient is under the hood. Furthermore, the cyclorotation of the eye due to the change from a sitting position to a laying position can make the pupil center change between the wavefront capture and the laser ablation, for example as discussed in Chernyak, D. A., J. Cataract. Refract. Surg., 30:633-638 (2004). Theoretically, it has been reported that correction of error due to rotation and translation of the pupil can provide significant benefits in vision correction. Certain aspects of these ocular effects are discussed in Bará, S. et al., Appl. Opt., 39:3413-3420 (2000) and Guirao, A. et al., J. Opt. Soc. Am. A, 18:1003-1015 (2001).

Intraocular Lens Implantation

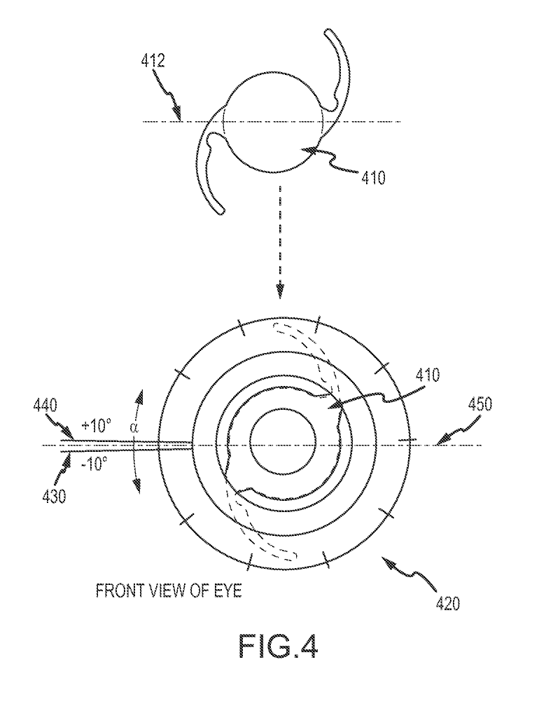

FIG. 4 depicts a front view of an intraocular lens 410 as implanted in an eye 420 of a patient. As shown here, the lens has an alignment axis 412. As part of an implantation procedure, one goal of the surgeon or physician is typically to align or orient the alignment axis 412 in a particular or target orientation 450 relative to the eye of the patient. Often, however, an implanted intraocular lens 410 will exhibit a rotation misalignment or error. Such misalignment or error can be associated with or a result of intraoperative physician placement of the lens. For example, as shown here, the intraocular lens can have a target orientation 450 relative to the patient eye, yet the implanted orientation of the intraocular lens may deviate from the target orientation 450. In some cases, the implanted orientation may deviate from the target orientation, within a rotational range between a negative limit 430 and a positive limit 440. In some cases, the negative limit 430 corresponds to a maximum negative intraoperative rotation of the lens (e.g. −10 degrees relative to the target orientation). In some cases, the positive limit 440 corresponds to a maximum positive intraoperative rotation of the lens (e.g. +10 degrees) relative to the target orientation. In some cases, the difference between the limits 430 and 440 can be about 20 degrees. Relatedly, in some cases a rotational range (e.g. between limits 430 and 440) can correspond to an arc of angular rotation having a value up to about 20 degrees. In some cases, the rotational range may be less than 20 degrees. In some cases, the rotational range can correspond to a 10 degree arc of angular rotation. In some cases, the rotational range can correspond to a 5 degree arc of angular rotation. In some cases, the negative limit 430 can have a value between 0 and −10 degrees. For example, the negative limit 430 can be −2.5 degrees, −5 degrees, and the like. In some cases, the positive limit 440 can have a value between 0 and +10 degrees. For example, the positive limit 440 can be +2.5 degrees, +5 degrees, and the like. Hence, in some cases, the negative limit 430 can be −5 degrees relative to an intended lens orientation or target orientation 450 and the positive limit 440 can be +5 degrees relative to the intended lens orientation or target orientation 450. In some cases, the error may refer to postoperative rotation instead of intraoperative rotation, or to postoperative rotation in combination with intraoperative rotation. For example, with regard to postoperative rotation, there may be a difference between rotation at baseline (e.g. at implantation) and a post-surgical date (e.g. 6 months).

Exemplary techniques for measuring IOL rotation (e.g. axis mark rotation) can be found in Patel et al., “Postoperative intraocular lens rotation: a randomized comparison of plate and loop haptic implants” Ophthalmology 106 (11) pp. 2190-2195 (1999), Weinand et al., “Rotational stability of a single piece hydrophobic acrylic intraocular lens: New method for high-precision rotation control” J. Cataract Refract. Surg. 33 pp. 800-803 (2007), and Praveen et al. “Software-based assessment of postoperative rotation of toric intraocular lens” J. Cataract Refract. Surg. 35 pp. 413-418 (2009), each incorporated herein by reference.

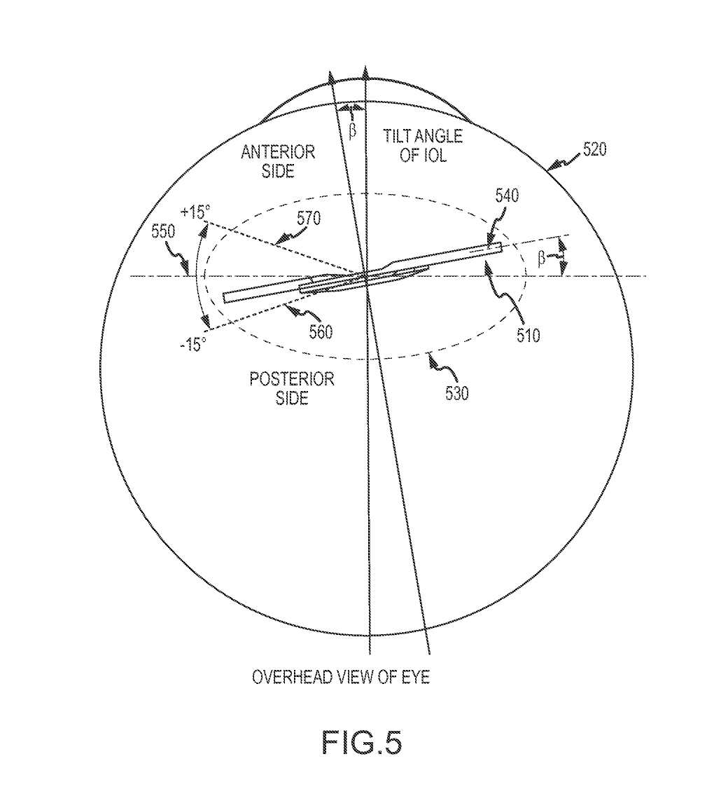

As shown in the overhead view of FIG. 5, an intraocular lens 510 can exhibit tilt upon implantation in an eye 520 of a patient (e.g. in the capsular bag 530). As part of an implantation procedure, one goal of the surgeon or physician is typically to align or orient the intraocular lens (e.g. the alignment axis 540 of the lens) in a particular or target orientation 550 relative to the eye of the patient. Often, however, an implanted intraocular lens will exhibit a tilt misalignment or error. Such misalignment or error can be associated with or a result of intraoperative physician placement of the lens. For example, as shown here, the intraocular lens can have a target orientation 550 relative to the patient eye, yet the implanted orientation of the intraocular lens may deviate from the target orientation 550. For example, the implanted orientation may deviate from the target orientation, within a tilt range between a negative limit 560 and a positive limit 570. In some cases, the negative limit 560 corresponds to a maximum negative intraoperative tilt of the lens (e.g. −7.5 degrees relative to the target orientation). In some cases, the positive limit 570 corresponds to a maximum positive intraoperative tilt of the lens (e.g. +7.5 degrees) relative to the target orientation. In some cases, the difference between the limits 560 and 570 can be about 15 degrees. Relatedly, in some cases a tilt range (e.g. between limits 560 and 570) can correspond to an arc of angular rotation having a value up to about 30 degrees. In some cases, the tilt range may be less than 30 degrees. In some cases, the tilt range can correspond to a 15 degree arc of angular tilt. In some cases, the tilt range can correspond to a 10 degree arc of angular tilt. In some cases, the tilt range can correspond to a 5 degree arc of angular tilt. In some cases, the tilt range can correspond to a 10 degree arc of angular rotation. In some cases, the tilt range can correspond to a 5 degree arc of angular rotation. In some cases, the negative limit 560 can have a value between 0 and −7.5 degrees. For example, the negative limit 560 can be −2.5 degrees, −5 degrees, and the like. In some cases, the positive limit 570 can have a value between 0 and +7.5 degrees. For example, the positive limit 570 can be +2.5 degrees, +5 degrees, and the like. Hence, in some cases, the negative limit 560 can be −5 degrees relative to an intended lens orientation or target orientation 550 and the positive limit 570 can be +5 degrees relative to the intended lens orientation or target orientation 550. In some cases, the negative limit corresponds to a maximum negative intraoperative tilt of the lens (e.g. −15 degrees relative to the target orientation). In some cases, the positive limit corresponds to a maximum positive intraoperative tilt of the lens (e.g. +15 degrees) relative to the target orientation. In some cases, the tilt angle β between the target orientation 550 and the implanted orientation can be within a range from about 0 degrees to about ±15 degrees. In some cases, the error may refer to postoperative tilt instead of intraoperative tilt, or to postoperative tilt in combination with intraoperative tilt. For example, with regard to postoperative tilt, there may be a difference between tilt at baseline (e.g. at implantation) and a post-surgical date (e.g. 6 months).

Exemplary techniques for measuring IOL tilt can be found in Tabernero et al. “Instrument for measuring the misalignments of ocular surfaces” Optics Express, 14 (22), 10945-10956 (2006) and Schaeffel “Binocular lens tilt and decentration measurements in healthy subjects with phakic eyes” Invest Ophthalmol Vis Sci, 49 (5), 2216-2222 (2008), both incorporated herein by reference. These papers illustrate the measurement techniques with the crystalline lens, and have been used, and are very suitable, for measuring IOL tilt as well.

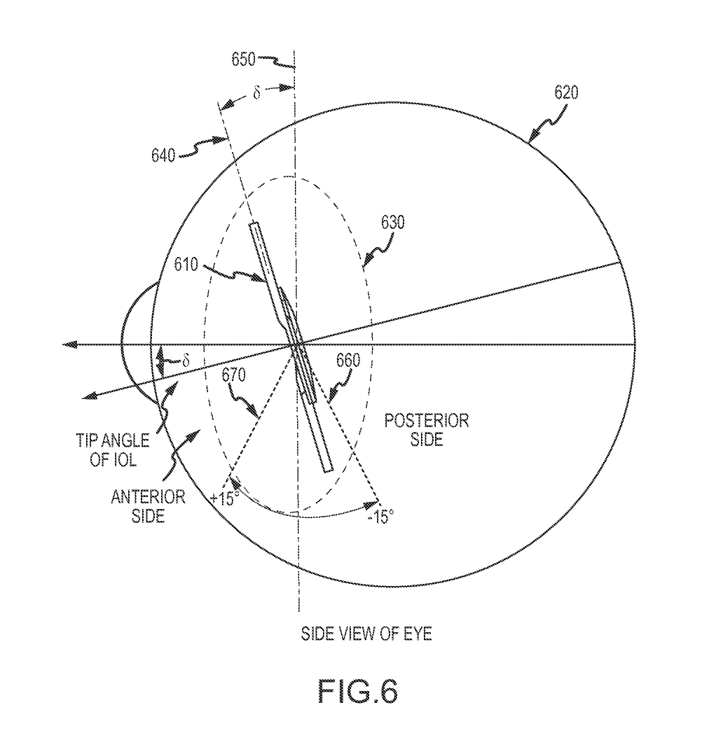

As shown in the side view of FIG. 6, an intraocular lens 610 can exhibit tip following implantation in an eye 620 of a patient (e.g. in the capsular bag 630). As part of an implantation procedure, one goal of the surgeon or physician is typically to align or orient the intraocular lens (e.g. the alignment axis 640 of the lens) in a particular or target orientation 650 relative to the eye of the patient. Often, however, an implanted intraocular lens will exhibit a tip misalignment or error. Such misalignment or error can be associated with or a result of intraoperative physician placement of the lens. In some cases, the misalignment or error can be a result of postoperative effects. For example, as shown here, the intraocular lens can have a target orientation 650 relative to the patient eye, yet the implanted orientation of the intraocular lens may deviate from the target orientation 650. For example, the implanted orientation may deviate from the target orientation, within a tip range between a negative limit 660 and a positive limit 670. In some cases, the negative limit 660 corresponds to a maximum negative intraoperative tip of the lens (e.g. −7.5 degrees relative to the target orientation). In some cases, the positive limit 670 corresponds to a maximum positive intraoperative tip of the lens (e.g. +7.5 degrees) relative to the target orientation. In some cases, the difference between the limits 660 and 670 can be about 15 degrees. Relatedly, in some cases a tip range (e.g. between limits 660 and 670) can correspond to an arc of angular rotation having a value up to about 30 degrees. In some cases, the tip range may be less than 30 degrees. In some cases, the tip range can correspond to a 10 degree arc of angular rotation. In some cases, the tip range can correspond to a 5 degree arc of angular rotation. In some cases, the negative limit 660 can have a value between 0 and −7.5 degrees. For example, the negative limit 660 can be −2.5 degrees, −5 degrees, and the like. In some cases, the positive limit 670 can have a value between 0 and +7.5 degrees. For example, the positive limit 670 can be +2.5 degrees, +5 degrees, and the like. Hence, in some cases, the negative limit 660 can be −5 degrees relative to an intended lens orientation or target orientation 650 and the positive limit 670 can be +5 degrees relative to the intended lens orientation or target orientation 650. In some cases, the negative limit corresponds to a maximum negative intraoperative tip of the lens (e.g. −15 degrees relative to the target orientation). In some cases, the positive limit corresponds to a maximum positive intraoperative tip of the lens (e.g. +15 degrees) relative to the target orientation. In some cases, the tip angle δ between the target orientation 650 and the implanted orientation can be within a range from about 0 degrees to about ±15 degrees. In some cases, the error may refer to postoperative tip instead of intraoperative tip, or to postoperative tip in combination with intraoperative tip. For example, with regard to postoperative tip, there may be a difference between tip at baseline (e.g. at implantation) and a post-surgical date (e.g. 6 months).

Often, the term tilt can refer to the collective combination of tilt and tip (e.g. tilt as depicted in FIG. 5 combined with tip as depicted in FIG. 6. As discussed elsewhere herein, tip and tilt can be associated with first order Zernike polynomials. For example, tip can be associated with a (second) Zernike coefficient c₂ and a Zernike polynomial Z₂, and tilt can be associated with a (third) Zernike coefficient c₃ and a Zernike polynomial Z₃. With regard to the combined geometric effect of tip and tilt, the combination can be described mathematically as:

Collective Tilt=\sqrt{(c₂²+c₃²)} (0)

where c₂ (tip) and c₃ (tilt) are the Zernike coefficients for the x- and y-components of the collective tilt. Throughout this disclosure, in certain instances the term tilt can refer to the collective tilt, and in certain instances the term tilt can refer to solely the tilt component of the collective tilt (which includes tilt and tip). According to some embodiments, collective tilt can also be characterized in terms of the δ and β angles described in FIGS. 5 and 6.

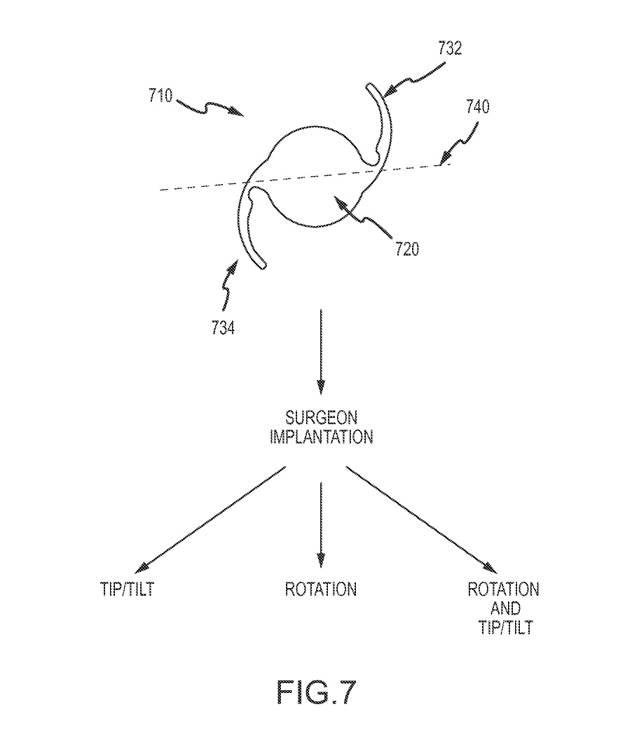

FIG. 7 depicts an intraocular lens 710 having a rotationally symmetric or asymmetric optical shape 720, two haptics 730a, 730b, and an axis 740. In some cases, the axis can be referred to as an alignment axis. The alignment axis can be a rotation alignment axis, a tip alignment axis, or a tilt alignment axis. In some cases, the axis can relate to the corrective quality of the lens. For example, the axis can be an anterior cylinder axis denoting a meridian with the lowest power. In some cases, the lens may have more than one axis. In some cases, an axis relating the corrective quality of the lens can be different from an axis used for alignment. In some cases, there is a known positional or alignment relationship between an axis relating the corrective quality of the lens and an axis used for alignment. In some cases, an axis relating to the corrective quality of the lens can also be used for alignment. In some cases, an axis relating to the corrective quality of the lens and/or an axis used for alignment can have a known relationship with the position, orientation, or configuration of a lens engagement means (e.g. haptics). In some cases, a lens engagement means can define an axis that corresponds to an axis relating to the corrective quality of the lens and/or an axis used for alignment. As shown here, malpositioning of the intraocular lens may occur in many implantation procedures.

Developing a Treatment Shape

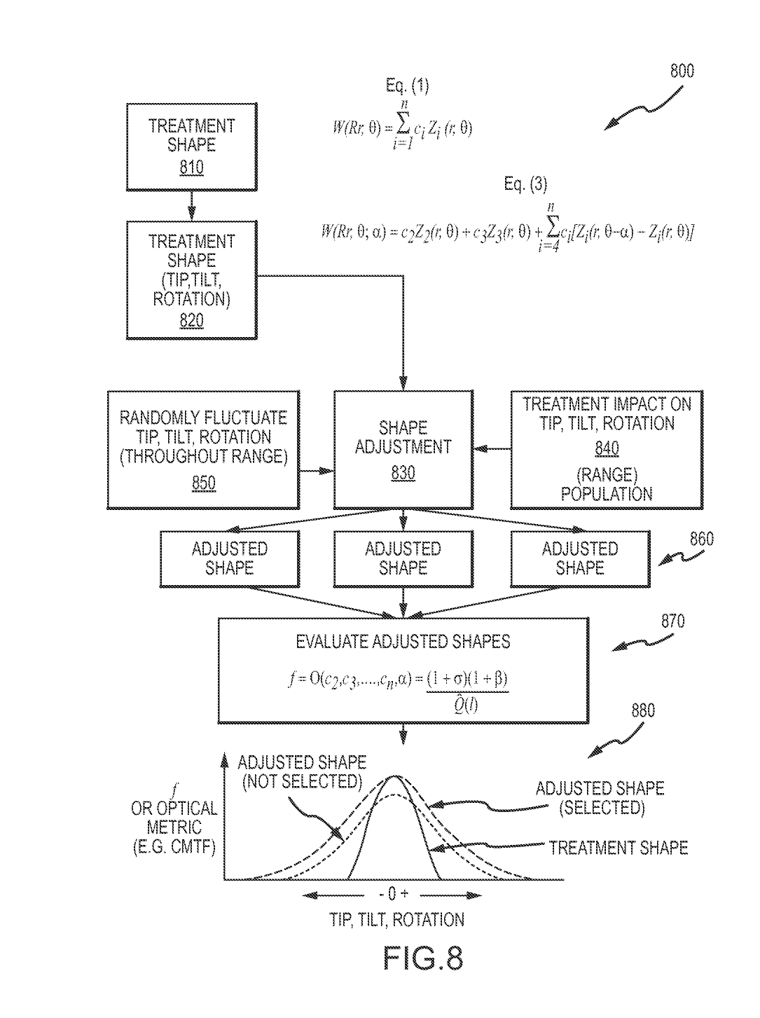

FIG. 8 depicts aspects of a treatment shape generation process 800 according to embodiments of the present invention. The process can be used to develop an intraocular treatment lens shape for treating an eye of a patient. The lens shape can be tolerable to intraoperative misalignment (or postoperative orientation effects) and provide quality vision to the patient. As shown here, the process may include obtaining a treatment shape 810, which can be characterized in terms of Zernike polynomials. The treatment shape 810 can correspond to an intraocular lens base shape, and can be represented by Eq. (1) as discussed elsewhere herein. The process can also include characterizing the treatment shape using the first order Zernike polynomial for tip and tilt. In some cases, the treatment shape can be characterized using a rotation term. In some cases, the treatment shape 820 can be characterized using the tip, tilt, and rotation terms. The treatment shape 820 can correspond to an intraocular lens base shape, and can be represented by Eq. (3) as discussed elsewhere herein.

Hence, the treatment shape or base shape (e.g. 810 or 820), which may be a rotationally symmetric optical shape, can be decomposed into various basis functions, such as power series, Zernike polynomials, Taylor monomials, and the like. In order to take into account of the effect of tip, tilt, and rotation for intraocular lenses (and other vision treatment modalities) it can be helpful to decompose the optical shape into a general two-dimensional basis function. For example, the optical shape or design can be expressed in terms of Zernike polynomials, as follows:

$$W ( Rr , θ ) = {\sum }_{i = 1}^{n} {c}_{i} {Z}_{i} ( r , θ ) \qquad ( 1 )$$

where R is the radius of the optical aperture, cᵢ is the ith Zernike coefficient, Zᵢ is the ith Zernike polynomial, and r and θ are polar coordinate variables. For example, r can be the distance of a variable point from the origin (e.g. radial variable in polar coordinates) and θ can be an angle between the positive x-axis and a vector corresponding to the point. According to some embodiments, r can be the normalized distance (from 0 to 1) of a variable point from the origin. Relatedly, r can be a dependent variable and θ can be an independent variable. By incorporating the θ term, it is possible to characterize rotational asymmetry. In contrast, if only r is used, it is possible to represent the two dimensional systems (e.g. the treatment shape, the wavefront, and the like) although the shape must be rotationally symmetric. By using both r and θ, it is possible to provide a full representation of any 2 dimensional surface, in a polar coordinate system. As illustrated by Eq. (1), it is possible to represent a wavefront (e.g. of an electromagnetic field) using a Zernike polynomial expression.

Additional surface expansion techniques (e.g. Fourier and discrete surface) are described in US 2014/0016091, the content of which is incorporated herein by reference. According to some embodiments, as an alternative to the Zernike expansion described in Eq. 1 above, such other surface expansion approaches can be implemented for characterizing treatment shapes, base shapes, and optical shapes as discussed herein.

The first term in Zernike polynomials is the piston term (zeroth order Zernike polynomial, Z₀⁰). Typically, the piston term does not have an optical effect and often can be ignored. The second Zernike term is tip (Y-tilt, first order Zernike polynomial, Z₁⁻¹) and the third Zernike term is tilt (X-tilt, first order Zernike polynomial, Z₁¹). Coefficients c₂ and c₃ can correspond to polynomials Z₂ (Y-tilt or Z₁⁻¹) and Z₃ (X-tilt or Z₁¹) respectively. Relatedly, aspects of IOL tilt are illustrated in FIG. 5, and aspects of IOL tip are illustrated in FIG. 6. Collectively, the second and third terms can be used to characterize or model the tilt effect of an optical correction. That is, in some embodiments, the term tilt can be used to refer to the combination of tip and tilt, as described in Eq. 0, above. Combining these first order terms (e.g. Z₁⁻¹ and Z₁¹) can provide a general equation for a plane, and the plane can have any orientation based on the values of the coefficients c₂ and c₃. As discussed elsewhere herein, these two terms collectively can represent a tilt in the wavefront.

For rotational effect, supposing the angle of rotation (which can also refer to or intraoperative or postoperative rotational error) is a, the optical surface will become

$$W ( Rr , θ ; α ) = {\sum }_{i = 1}^{n} [ {c}_{i} {Z}_{i} ( r , θ - α ) - {g}_{i} {Z}_{i} ( r , θ ) ] \qquad ( 2 )$$

where gᵢ corresponds to the Zernike coefficients for a reference wavefront, or a wavefront without rotational error. According to some embodiments, cᵢ corresponds to Zernike coefficients ro a wavefront with a rotational error of a angle. Relatedly, aspects of IOL rotation are illustrated in FIG. 4. For pure rotation/tilt effect, we have gᵢ=cᵢ for i=4:n. Therefore, Eq (2) can also be rewritten as

$$W ( Rr , θ ; α ) = {c}_{2} {Z}_{2} ( r , θ ) + {c}_{3} {Z}_{3} ( r , θ ) + {\sum }_{i = 4}^{n} {c}_{i} [ {Z}_{i} ( r , θ - α ) - {Z}_{i} ( r , θ ) ] \qquad ( 3 )$$

Hence, Eq. (3) can be used to characterize an optical surface in a way that incorporate the angle of rotation a. According to some embodiments, rotational error as characterized by α can be implemented as a constant. In some cases, rotational error as characterized by α can vary, for example based on results associated with a number of intraocular lenses. For example, given 100 implanted intraocular lenses, the first lens may have a 2.3 degree rotational error, the second lens may have a 3.1 degree rotational error, and so on. In some cases, results associated with a number of implanted lenses can be used to determine a constant value for α. Similarly, according to some embodiments, tip and tilt as characterized by c₂ (tip) and c₃ (tilt) can be implemented as constant values. In some cases, tip or tilt error as characterized by c₂ (tip) and c₃ (tilt) can vary, for example based on results associated with a number of intraocular lenses. For example, given 100 implanted intraocular lenses, the first lens may have a 2.3 degree tip or tilt error, the second lens may have a 3.1 degree tip or tilt error, and so on. In some cases, results associated with a number of implanted lenses can be used to determine constant values for c₂ (tip) and c₃ (tilt).

By introducing the θ variable (e.g. angle between the positive x-axis and a vector corresponding to the point corresponding to r), it is possible to incorporate the effect of rotation, and asymmetric shapes. Relatedly, embodiments of the present invention are unique in that they can address rotational error via the θ and/or α variables as discussed herein. Further, embodiments of the present invention are unique in that they can address other errors via the c₁, c₂, and/or c₃, variables. Yet further, embodiments of the present invention are unique in that they can address other errors via rotationally asymmetric terms such as tip and tilt (e.g. instead of or in addition to rotationally symmetrical terms (e.g. defocus, primary spherical aberration, and secondary spherical aberration.

In general, low order aberrations include first-order aberrations such as piston (plano) and prism (tilt) as well as second-order aberrations such as sphere (defocus) and cylinder, and aberrations higher than the second-order are considered high order aberrations. According to some embodiments, techniques disclosed herein can involve considering high order Zernike terms (e.g. up to sixth-order). In some cases, apart from the tip and tilt, the two terms in the summation (i=4 to n) in Eq (3) represent the actual wavefront (e.g. having a rotational error of a) and the reference wavefront (having no rotational error).

With returning reference to FIG. 8, the process 800 can also include obtaining an intraocular lens implantation effect corresponding to an operator influence profile. For example, the implantation effect can correspond to a treatment impact 840 on tip, tilt, collective tilt, rotation, or a subcombination thereof. The impact or influence profile 840 can be based on a population of surgeons, physicians, or operators. Throughout this disclosure, the terms surgeon, physician, and operator can be used interchangeably, according to some embodiments. In some cases, the impact 840 can correspond to a range. For example, the impact 840 can correspond to or include a rotation range between −10 degrees and +10 degrees (e.g. FIG. 4). Similarly, the impact 840 can correspond to or include a tilt range between −15 degrees and +15 degrees (e.g. FIG. 5). Relatedly, the impact 840 can correspond to or include a tip range between −15 degrees and +15 degrees (e.g. FIG. 6). In some cases, the impact 840 can refer to the collective tilt (e.g. the tilt as depicted in FIG. 5 combined with the tip as depicted in FIG. 6).

An example of measured IOL tilt and related standard deviation in the measured population is discussed in Mester et al., “Decentration and tilt of a single piece aspheric intraocular lens compared with the lens position in young phakic eyes” J Cataract Refract Surg, 35 (3), 485-490 (2009), incorporated herein by reference. Exemplary measurement values include: tip (2.5±1.4 degrees) and tilt (2.6±1.5 degrees). To include the IOL tilt of 95% of the population (±2 standard deviations), the ranges of −0.3 to +5.3 degrees for tip and −0.4 to 5.6 degrees for tilt, for example, can be used in the treatment shape generation techniques discussed herein. An example of measured IOL rotation is discussed in TECNIS Toric IOL Foldable Posterior Chamber Intraocular Lenses [package insert], Santa Ana, Calif.: Abbott Medical Optics Inc. 3 ANSI Z80.30-2010, Approved Mar. 24, 2010, incorporated herein by reference. Exemplary measurement values include: rotation (2.74±5.65 degrees). To include the IOL rotation of 95% of the population (±2 standard deviations), the range of −8.5 to +14 degrees for rotation, for example, can be used in the treatment shape generation techniques discussed herein.

Although physicians are typically very precise when implanting intraocular lenses or delivering other vision treatment modalities, some implantation error may occur. As an example, a doctor may implant ten intraocular lenses in ten patient eyes, and the mean rotational error may be 0 degrees, although individual lenses within the ten lenses may exhibit some amount of rotational error. In some cases, each of the ten implanted lenses may exhibit some amount of rotational error. The rotation error (or other placement error such as tip error, tilt error, or collective tilt error) can occur as a probability distribution. For example, there may be a higher number of occurrences of low rotational error (e.g. rotational error is close to 0) and a lower number of occurrences of high rotational error (e.g. rotational error is much less than or much greater than 0). According to some embodiments, it is possible to generate the set of adjusted shapes based on such a probability distribution.

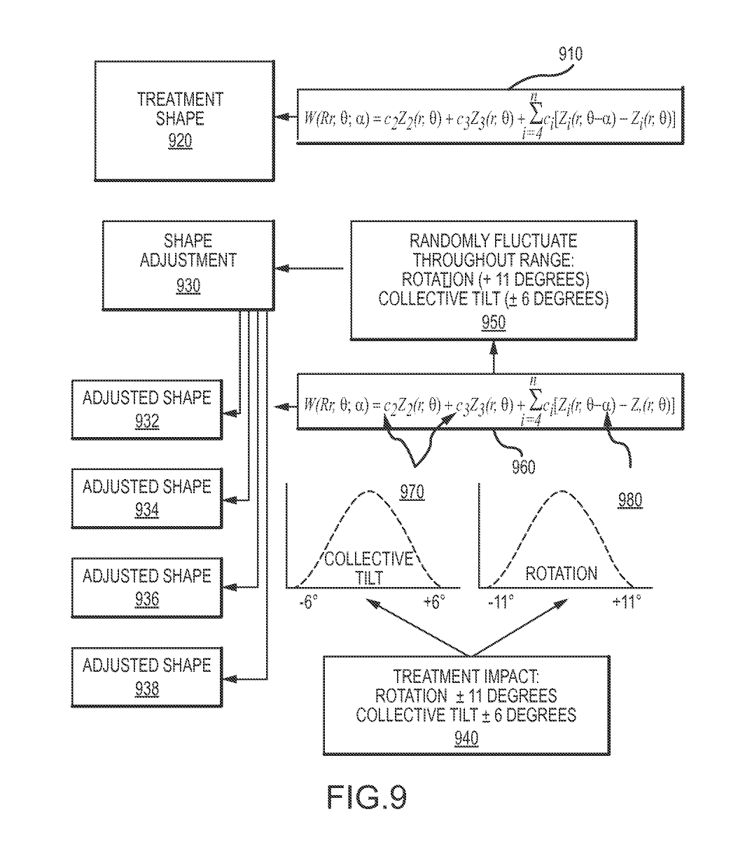

The process 800 can also include a shape adjustment protocol 830, whereby aspects (e.g. tip, tilt, or rotation) of the treatment shape or base shape 820 are randomly fluctuated throughout the range associated with impact 840, such that a set of adjusted shapes 860 is produced. According to some embodiments, the shape adjustment and selection process can involve randomly varying the variables (e.g. angles α, β, and/or δ as depicted in FIGS. 4, 5, and/or 6) within their respective range per the probability distribution function (e.g. distributions 970, 980 as depicted in FIG. 9), and the evaluation protocol 870 or optimization algorithm can be used to determine the optical metric or merit function value, and the process can be continued or iterated until the change in the optical metric or merit function value is smaller than a predefined value, in which case the evaluation or optimization converges and the process stops. Individual adjusted shapes of the set of adjusted intraocular lens shapes can be based on the random fluctuation of the base shape, and the set can be based on the implantation effect or treatment impact 840. In some cases, the base shape aspects can be randomly fluctuated according to a normal distribution. For example, there may be a higher probability that the fluctuated variable will be closer to 0 rotation (or tip or tilt, or collective tilt).

In some cases, the random fluctuation protocol 850 can involve using random numbers for c₂, c₃, and α (e.g. for Eq. 3) so as to construct random wavefront shapes or optical shapes for the set of adjusted shapes 860.

According to some embodiments, it is possible to vary certain variables of Eq. 3 (e.g. c₂, c₃, a, c₄, c₅, . . . c₂₇, and so on). For example, selected variables (e.g. c₂, c₃, and/or a) can be varied according to a certain probability function (e.g. distributions 970, 980 of FIG. 9) and other variables (e.g. c₄, . . . , c₂₇) can be varied according to a purely random fluctuation (e.g. based on an optimization algorithm). In this way, it is possible to generate a set of numbers for the first group of variables (e.g. c₂, c₃, and α) and randomly fluctuate a set of numbers for the second group of variables (e.g. c₄, . . . , c₂₇) to obtain an optimized shape. It is then possible to select another set of c₂, c₃, and α, and optimize the shape. Eventually, it is possible to obtain an optimized shape per a set of c₂, c₃, and a. Relatedly, for each set of the three numbers (c₂, c₃, and a) it is possible to construct an intraocular lens with a certain optical shape, in some case based on the wavefront calculation according to Eq. 3.

According to some embodiments, a narrower range (of error in e.g. c₂, c₃, and α) can produce a more accurate optimized shape, and a wider range (of error in e.g. c₂, c₃, and α) can produce a relatively less accurate optimized shape. Hence, the quality of the adjusted shapes can be a function of the range.

According to some embodiments, the merit function or optical metric protocol can involve establishing an optically optimized shape appropriate for a vision condition, where the merit function or optical metric is optimized. For example, a Strehl ratio, encircled energy, MTF, MTF volume or volume under MTF surface (MTFV), compound modulation transfer function (CMTF), or contrast sensitivity (CS) can be maximized, and in some cases it is also desirable to minimize the fluctuation of the merit function or optical metric, for example as described in US 2014/0016091 the content of which is incorporated herein by reference.

As depicted in FIG. 8, methods may include adjusting the treatment shape, as indicated at step 830. The adjustment technique can be based on treatment impact 840 and shape fluctuation 850. Following a vision treatment protocol, however, it is often observed that the aspects of the treatment may have an impact 840 on the outcome with regard to tip, tilt, and/or rotation. For example, administration of the treatment can introduce or result in some amount of tip or tilt. Such lens orientations can introduce or amplify certain optical effects like coma, prism, and the like, which may be undesired. Such orientation impacts may depend on the experience of the surgeon or person administering the treatment. Relatedly, the impacts can be characterized in terms of populations of surgeons. In the case of intraocular lens treatments, the lens is typically secured in the patient's eye using lens haptics or other attachment or engagement means. In some cases, the surgeon may not achieve a completely precise implantation of the lens. For example, the lens may not be accurately secured to or positioned relative to the capsular bag or other anatomical features within the eye. In some cases, the treatment may require placing haptics within the capsular bag or attaching haptics to the capsular bag or lens capsule, and the placement or attachment may not be performed as desired. Misalignments, including rotation and tip and/or tilt may result. For example, a population of surgeons may be associated with postoperative rotation or intraoperative rotational placement error of the implanted lens within a range from −5° to +5° off axis.

Hence, the optical shape can be determined as given in Eq. (3), a set of adjusted shapes 860 can be produced, an optical metric such as a compound modulation transfer function (CMTF) can be calculated, and the optical metric can be processed with a merit function or goal function.

For example, the process 800 can include evaluating the adjusted shapes of the set 860, as depicted at step 870. In some cases, the adjusted shapes can be evaluated according to an optical metric and a merit function. Relatedly, a merit function or goal function value (e.g. ƒ) can also be calculated. As an example of an optical metric, a compound modulation transfer function (CMTF) may be calculated as

$${C}^{( m )} ( l ) = \frac{1}{m} {\sum }_{i = 1}^{m} {γ}_{i} {M}_{i} ( l ) \qquad ( 4 )$$

where m is the number of spatial frequencies used for the calculation of MTF, l is the vergence, or the reciprocal of the vision testing distance (in meters), M is the MTF, and γ is the reciprocal of the diffraction-limited MTF at the specific spatial frequency.

In some cases, the merit function or goal function can relate to optical quality, and it can be, for example, based on, or a function of (or related to) optical metrics such as Strehl ratio (SR), modulation transfer function (MTF), point spread function (PSF), encircled energy (EE), MTF volume or volume under MTF surface (MTFV), or contrast sensitivity (CS); and optionally to new optical metrics which are appropriate to vision conditions such as presbyopia; for instance, compound modulation transfer function (CMTF) as described elsewhere herein. In optical terms, the merit function or goal function should make sense. That is to say, minimization or maximization of the merit function or goal function should give a predictable optimized optical quality of the eye. The goal function can be a function with a certain number of free parameters to be optimized (minimized) through an optimization, or minimization, algorithm. According to some embodiments, an optimization or minimization algorithm can incorporate a downhill simplex method, a direction set method, a simulated annealing method, or the like. As discussed elsewhere herein, an evaluation protocol or optimization algorithm can be used to determine the optical metric or merit function value, and the process can be continued or iterated until the change in the optical metric or merit function value is smaller than a predefined value or converges to a predefined accuracy.

Although there are many types of goal functions available for use with the present invention, the discussion below generally touches on two broad schools of goal functions. In a Diffraction Theory based approach, the shape is considered as a wave aberration. Typically, a Fourier transform is employed for calculating optical quality related parameters, such as Strehl ratio (SR), modulation transfer function (MTF), MTF volume or volume under MTF surface (MTFV), compound modulation transfer function (CMTF), or contrast sensitivity (CS), encircled energy (EE) (based on point spread function), as well as special cases that combine one or more of these parameters, or values of the parameters in specific situations (such as MTF at spatial frequency or encircled energy at a field of view), or integration of any parameters (volume of MTF surface at all frequencies or up to a cutoff frequency, for example 60 cycles/degree or 75 cycles/degree, because 60 cycles/degree is the retina cone's limiting spatial frequency). In a Geometrical Optics approach, or the so-called ray tracing approach, the optical effect is based on ray tracing. With both the Diffraction Theory and the Geometrical Optics approaches, polychromatic point spread function with Stiles-Crawford effect, chromatic aberrations as well as retina spectral response function can be used.

In some cases, the optical metric can be a point spread function, such as a monochromatic point spread function. The point spread function of an optical system or shape can be based on the power spectrum of the Fourier transform of the generalized pupil function. In some cases, a polychromatic point spread function can be used as an optical metric, for example because of chromatic aberrations and the Stiles-Crawford effect in the human eye. In some cases, another Fourier transform can be performed to obtain a modulation transfer function.

Monochromatic point spread function (PSF) has been used for describing optical defects of optical systems having aberrations. Due to the simple relationship between wave aberrations and the PSF for an incoherent light source, Fourier transform of the generalized pupil function has been used in the calculation of point spread functions. Most optical applications, however, do not use a monochromatic light source. In the case of human vision, the source is essentially white light. Thus, there may be limitations associated with the use of monochromatic PSF as a goal function.

Polychromatic point spread function (PSF) with correct chromatic aberrations, Stiles-Crawford effect as well as retina response function, can be used for optical modeling of human eyes. Here, chromatic aberrations arise because light composed of different wavelengths will focus either in front of the retina or behind it. Only portions of the light will focus exactly on the retina. This gives the eye an extended depth-of-focus, i.e., if one has focusing error of some amount, the eye is still capable of focusing at least for some wavelengths. Therefore, chromatic aberrations in fact help the correction of presbyopia. If the depth-of-focus is sufficiently large, there would be no presbyopia problem. Unfortunately, the chromatic aberrations are not large enough and it also varies with the wavelength. Stiles-Crawford effect, also known as pupil apodization, is due to the waveguide property of the retinal cones. Light from the pupil periphery has a slightly less chance of being detected by the retina because the ray of light might not reach the bottom of the cone, due to a slight incident angle. As for the retinal spectral response function, it is known that the cones, which are responsible for daylight vision, have different sensitivity to different wavelengths. Only green light is absorbed by the eye almost completely. Both blue light and red light are absorbed by the eye partially.

Once the PSF is calculated, calculation of the Strehl ratio is straightforward. Strehl ratio can be defined as the ratio of the peak of the point spread function (PSF) of an optical system to the peak of a diffraction-limited optical system with the same aperture size. A diffraction-limited optical system is typically a system with no aberrations, or optical errors. It can be an ideal or perfect optical system, having a Strehl ratio of 1.

As discussed in US Patent Publication No. 2014/0016091, the content of which is incorporated herein by reference, such CMTF equations can provide a measure of optical quality for the shape throughout a range of vergence or at different spatial distances (l).

As noted above, Eq. 4 can provide a set of numbers as a function of 1, (e.g. CMTF values at different distances/vergence). Hence, if the distances values are selected for every 0.1 Diopter or 0.1 meter, and 3.0 Diopters of vergence is desired, then there will be 30 data points and 30 values for C (optical metric value). Based on those 30 values, it is possible to calculate mean, standard deviation, and peak-to-valley for Eq. 5.

The merit function value may be calculated as:

$$f = O ( {c}_{2} , {c}_{3} , … , {c}_{n} , α ) = \frac{( 1 + σ ) ( 1 + β )}{\hat{Q} ( l )} \qquad ( 5 )$$

where \hat{Q}(l), σ, and β are the mean, standard deviation, and peak-to-valley (maximum to minimum), respectively, of the optical metric given in Eq. (4) for CMTF or other metrics like Strehl Ratio (SR), modulation transfer function (MTF), point spread function (PSF), encircled energy (EE), MTF volume or volume under MTF surface (MTFV), contrast sensitivity (CS), or various combinations thereof. Exemplary metrics are described in U.S. patent application Ser. No. 13/732,124 filed Dec. 31, 2012, the content of which is incorporated herein by reference.

According to some embodiments, once an optical metric is defined (e.g. Eq. 4), a merit function can be constructed so that a function minimization algorithm can be applied to maximize the optical metric. In some cases, the merit function is inversely proportional to the optical metric. In some cases, it is possible to maximize the optical metric while ensuring the optical metric curve over the range does not fluctuate excessively. An exemplary merit function or optimizer value is provided by Eq. 5.

As shown in the graph 880 in FIG. 8, it is possible to compare the merit function values of multiple adjusted shapes (e.g. of set 860) with one another, or to compare the merit function values of one or more adjusted shapes (e.g. of set 860) with the merit function values of a treatment shape that is not adjusted (e.g. shape 810 or 820). For example, with CMTF the vergence (l) can be fixed, and the CMTF values can be calculated throughout a range (e.g. range of rotation or angular values), and the merit function for optical metric (e.g. CMTF) can be plotted as a function of the rotational angle. As depicted here, the merit function for optical metric (e.g. CMTF) values for an original treatment shape (810 or 820) can have a central peak (e.g. at rotation angle 0) and diminish more rapidly as the rotation angle becomes more negative or more positive. According to some embodiments, the angle 0 is associated with the population mean. Similarly, the merit function ƒ for optical metric (e.g. CMTF) values for an adjusted treatment shape (e.g. of set 860) can have a central peak (e.g. at rotation angle 0) and diminish less rapidly as the rotation angle becomes more negative or more positive. Often, the adjusted shapes will provide a flatter or broader curve relative to the non-adjusted shape.

Hence, the adjusted shapes can exhibit a greater tolerability to rotational error (or other implantation impacts) as compared to the non-adjusted shape which exhibits less tolerability to the error. For example, adjusted shapes can be selected based on which shape exhibits a higher degree of tolerability to rotation, tip, or tilt (or combined tilt). As shown in graph 880, it is possible to select an adjusted shape based on the rate at which the merit function or optical metric value diminishes when offset from the target orientation. Similarly, it is possible to selected an adjusted shape based on the peak value of the merit function or optical metric. In some cases, it is possible to select an adjusted shape based on a combination of (i) the rate at which the merit function or optical metric value diminishes when offset from the target orientation, and (ii) the peak value of the merit function or optical metric. The range of tip, tilt, or rotation depicted on the x-axis of graph 880 can correspond to the range of physician error. For example, as discussed elsewhere herein, the rotational range of error can be between −10 degrees and +10 degrees. In this way, it is possible to determine an adjusted shape that provides desired vision characteristics throughout the physician error range. The selected adjusted shape, which can be based on population statistics, confers a good optical quality or visual outcome (e.g. visual acuity) to most patients, and tolerates misalignment or misplacement of the lens by most physicians.

As an example, the treatment shape (810 or 820) can be a shape for treating myopia, and the treatment impact (840) can include a physician error of ±5 degrees rotational error and a collective tilt error of ±2 degrees. It is possible to randomly fluctuate variables of the treatment shape in terms of the rotational and tilt offset ranges (e.g. randomly fluctuate the variables such as c₂, c₃, and a throughout the error range) so as to produce a set of adjusted shapes, and evaluate the adjusted shapes based on an optical metric, optionally in combination with a merit function. In some cases, the random fluctuation can be implemented according to a normal distribution, a Gaussian distribution, or some other distribution. For example, a set of 1000 adjusted shapes can be generated and evaluated according to the evaluation protocol (870), and a single adjusted optical shape can be selected (as depicted in graph 880), such that the selected adjusted shape exhibits a high degree of tolerability to changes in rotation and collective tilt.

As depicted in graph 880, when the merit function or optical metric value is calculated, it may be desirable to obtain a smooth variation of the merit function or optical metric (such as CMTF, Strehl ratio, and the like) over a range of tilt angles or rotational angles. It is possible to use, for example, the equation of the evaluation 870, and sigma (σ) can be the standard deviation of the values of the merit function or optical metric over the tilt angles and the beta (β) can be the PV of the variation. A similar protocol can be implemented for the rotational angle. When both tilt (e.g. collective tilt) and rotation factors are considered, it is possible to implement a weighting function for both factors. For example, the tilt and rotation factors can be equally weighted (50%-50%). In some cases, one of the factors may be weighted more heavily than the other. For example, for a high cylinder case, a rotational error may be more important than the tilt factor. Hence, the weighting factor for the rotational error may be larger than the weighting factor for tilt error. According to some embodiments, an algorithm can be used to determine the smoothness of the merit function or optical metric curve. In some cases, the smoothness can be determined based on a value as defined by the formula. In some cases, once it is optimized (a minimum optimizer value is found), it is possible to end the optimization and determine the optimized shape is acceptably invariant to the change of tilt and/or rotation of the intraocular lens implantation.

As noted above, any of a variety of optical metrics can be used for evaluating the adjusted shapes (or non-adjusted shapes), for example, SR, MTF, PSF, EE, MTFV, CS, or various combinations thereof.

Once an adjusted shape is selected based on the evaluation 870, it is possible to generate an intraocular lens treatment shape based on the selected shape. In some cases, the intraocular lens treatment shape is equivalent to the selected shape.

FIG. 9 depicts aspects of a shape adjustment protocol according to embodiments of the present invention. As illustrated here, a treatment shape 920 can be characterized in terms of a basis function, as indicated by step 910. In this example, the treatment impact 940 involves a rotation error range of ±11 degrees and a collective tilt error range of ±6 degrees. According to step 950, collective tilt variables (e.g. c₂, c₃) of the basis function 960 can be randomly fluctuated according to a distribution 970 throughout the collective tilt error range, and a rotation variable (e.g. α) of the basis function 960 can be randomly fluctuated according to a distribution 980 throughout the rotation error range. For example, the collective tilt variables can be fluctuated throughout a range corresponding to the collective tilt error range of treatment impact 940, and the rotation variable can be fluctuated throughout a range corresponding to the rotation error range of treatment impact 940. The shape adjustment step 930 involves generating multiple adjusted shapes 932, 934, 936, 938, and so on, based on the random fluctuations. An adjusted shape can then be selected based on an optical metric or merit function, as described elsewhere herein.

In some cases, selection of the adjusted shape may be influenced by the type of treatment lens. For example, undesired rotation may have a disproportionate impact for toric intraocular lenses that involve cylindrical correction. Hence, it may be particularly important to have appropriate rotational alignment of the cylindrical axis in a toric lens or to tolerate rotational misalignment of the lens. In some cases, undesired rotation may have less of an impact for intraocular lenses that involve only spherical correction, as such lenses are often rotationally symmetrical. Often, however, intraocular lenses involve an optical surface shape or vision treatment characteristic that is impacted by rotation of the lens.

The methods and apparatuses of the present invention may be provided in one or more kits for such use. The kits may comprise a system for profiling an optical surface, such as an optical surface of an eye, and instructions for use. Optionally, such kits may further include any of the other system components described in relation to the present invention and any other materials or items relevant to the present invention. The instructions for use can set forth any of the methods as described above.

Each of the calculations or operations described herein may be performed using a computer or other processor having hardware, software, and/or firmware. The various method steps may be performed by modules, and the modules may comprise any of a wide variety of digital and/or analog data processing hardware and/or software arranged to perform the method steps described herein. The modules optionally comprising data processing hardware adapted to perform one or more of these steps by having appropriate machine programming code associated therewith, the modules for two or more steps (or portions of two or more steps) being integrated into a single processor board or separated into different processor boards in any of a wide variety of integrated and/or distributed processing architectures. These methods and systems will often employ a tangible media embodying machine-readable code with instructions for performing the method steps described above. Suitable tangible media may comprise a memory (including a volatile memory and/or a non-volatile memory), a storage media (such as a magnetic recording on a floppy disk, a hard disk, a tape, or the like; on an optical memory such as a CD, a CD-R/W, a CD-ROM, a DVD, or the like; or any other digital or analog storage media), or the like.

All patents, patent publications, patent applications, journal articles, books, technical references, and the like discussed in the instant disclosure are incorporated herein by reference in their entirety for all purposes.

While the above provides a full and complete disclosure of the preferred embodiments of the present invention, various modifications, alternate constructions and equivalents may be employed as desired. Therefore, the above description and illustrations should not be construed as limiting the invention, which can be defined by the appended claims.

1. A method of generating an intraocular treatment lens shape for treating an eye of a patient, the method comprising:
obtaining an intraocular lens implantation effect corresponding to an operator influence profile;
obtaining an intraocular lens base shape;
producing a set of adjusted intraocular lens shapes, wherein individual adjusted shapes of the set are based on a random fluctuation of the base shape and the set is based on the implantation effect;
evaluating the adjusted shapes of the set according to a merit function;
selecting one of the adjusted shapes based on the evaluation; and
generating the intraocular lens treatment shape based on the selected shape.

2. (canceled)

3. The method according to claim 1, wherein the intraocular lens implantation effect comprises a rotation effect.

4. The method according to claim 3, wherein the rotation effect corresponds to a rotational range.

5.-7. (canceled)

8. The method according to claim 1, wherein the intraocular lens implantation effect comprises a tilt effect.

9. The method according to claim 8, wherein the tilt effect corresponds to a tilt range.

10.-12. (canceled)

13. The method according to claim 1, wherein the operator influence profile corresponds to a physician population.

14. The method according to claim 1, wherein the merit function comprises a parameter selected from the group consisting of Strehl Ratio (SR), modulation transfer function (MTF), point spread function (PSF), encircled energy (EE), volume under MTF surface (MTFV), compound modulation transfer function (CMTF), and contrast sensitivity (CS).

15. A system for generating an intraocular treatment lens shape for treating an eye of a patient, the system comprising:
a processor; and
a tangible non-transitory computer readable medium comprising a computer application that, when executed by the processor, causes the processor to:
access an intraocular lens implantation effect corresponding to an operator influence profile;
access an intraocular lens base shape;
produce a set of adjusted intraocular lens shapes, wherein individual adjusted shapes of the set are produced based on a random fluctuation of the base shape and the set is produced based on the implantation effect;
evaluate the adjusted shapes of the set according to a merit function;
select one of the adjusted shapes based on the evaluation; and
generate the intraocular lens treatment shape based on the selected shape.

16. (canceled)

17. The system according to claim 15, wherein the intraocular lens implantation effect comprises a rotation effect.

18. The system according to claim 17, wherein the rotation effect corresponds to a rotational range.

19.-21. (canceled)

22. The system according to claim 15, wherein the intraocular lens implantation effect comprises a tilt effect.

23. The system according to claim 22, wherein the tilt effect corresponds to a tilt range.

24.-26. (canceled)

27. The system according to claim 15, wherein the operator influence profile corresponds to a physician population.

28. The system according to claim 15, wherein the merit function comprises a parameter selected from the group consisting of Strehl Ratio (SR), modulation transfer function (MTF), point spread function (PSF), encircled energy (EE), volume under MTF surface (MTFV), compound modulation transfer function (CMTF), and contrast sensitivity (CS).

29. A computer product embodied on a tangible non-transitory computer readable storage medium, the computer product comprising:
code for accessing an intraocular lens implantation effect corresponding to an operator influence profile;
code for accessing an intraocular lens base shape;
code for producing a set of adjusted intraocular lens shapes, wherein individual adjusted shapes of the set are produced based on a random fluctuation of the base shape and the set is produced based on the implantation effect;
code for evaluating the adjusted shapes of the set according to a merit function;
code for selecting one of the adjusted shapes based on the evaluation; and
code for generating the intraocular lens treatment shape based on the selected shape.

30. (canceled)

31. The computer product according to claim 29, wherein the intraocular lens implantation effect comprises a rotation effect.

32. The computer product according to claim 31, wherein the rotation effect corresponds to a rotational range.

33.-35. (canceled)

36. The computer product according to claim 29, wherein the intraocular lens implantation effect comprises a tilt effect.

37. The computer product according to claim 36, wherein the tilt effect corresponds to a tilt range.

38.-40. (canceled)

41. The computer product according to claim 29, wherein the operator influence profile corresponds to a physician population.

42. The computer product according to claim 29, wherein the merit function comprises a parameter selected from the group consisting of Strehl Ratio (SR), modulation transfer function (MTF), point spread function (PSF), encircled energy (EE), volume under MTF surface (MTFV), compound modulation transfer function (CMTF), and contrast sensitivity (CS).
